(19) United States
(12) Patent Application Publication
(10) Pub. No.: US 2025/0375601 A1
(43) Pub. Date: Dec. 11, 2025

(54) TATTOO MACHINE

(71) Applicant: Thomas Sinnamond, Burien, WA (US)

(72) Inventor: Thomas Sinnamond, Burien, WA (US)

(21) Appl. No.: 19/224,720

(22) Filed: May 31, 2025

Related U.S. Application Data
(60) Provisional application No. 63/656,300, filed on Jun. 5, 2024.

(51) Int. Cl. A61M 37/00

(52) U.S. Cl. CPC A61M 37/0084

(57) ABSTRACT

A tattoo machine is provided. The tattoo machine includes an external casing with a top cover and a bottom cover. The tattoo machine includes a sleeve that partially encircles the bottom cover. The sleeve is connected to the bottom cover. A fluid bottle is removably positioned on the top of the sleeve. The tattoo machine includes a fluid dispensing mechanism having a tubing and a peristaltic pump. A tattoo needle is slidably disposed of in a tattoo nozzle. The fluid dispensing mechanism is configured to dispense a metered quantity of fluid from the removable fluid bottle to the tattoo needle in a controlled and contactless manner. A driving mechanism is configured to selectively operate the pump and enable longitudinal stroke motion for the tattoo needle.

### CROSS-REFERENCE TO RELATED APPLICATIONS

The present application hereby claims priority to and incorporates by reference the entirety of the disclosures of the provisional application No. 63/656,300, entitled “Tattoo Machine” filed on Jun. 5, 2024.

### FIELD OF THE INVENTION

The present invention relates to the field of tattoo machines, and more particularly, the present invention relates to a tattoo machine that includes a removable ink bottle and an ink dispensing mechanism to dispense a metered quantity of ink from the removable ink bottle to the tattoo needle in a controlled manner. The present invention is aimed at increasing both the speed and accuracy of the tattoo-making process.

### BACKGROUND

A tattoo machine, also referred to as a tattoo gun, is a hand-held portable device that is generally used to create a tattoo marking on a surface such as the skin of a user or an article using ink. In particular, a pin or needle punctures the surface and places the ink below the surface. Generally, the needle can reciprocate back and forth to puncture the surface. Various types of tattoo machines enable reciprocating movement of the needle back and forth to puncture the surface such as but not limited to a coil tattoo machine and a rotary tattoo machine. The coil tattoo machines are generally powered by a wired regulated DC power supply which sends an electric current through the copper coils wrapped around opposing magnets and then moves the armature bar up and down. The rotary tattoo machines are operated with regulated rotary motors and are powered by a wired external RC power supply or a wireless battery pack attached to the machine.

Generally, the needle of the tattoo machine needs to be repeatedly submerged (dipped) in an independent ink reservoir for ink refilling. The needle can be periodically dipped in the ink pigment, or the needle can reciprocate in contact with a reservoir of ink pigment to coat the ink pigment onto the needle. Repeated dipping takes time, breaks concentration, requires mental and physical effort, and is not perfectly repeatable. Eliminating the dipping step increases both speed and accuracy of the tattoo-making process. Further, the process of repeatedly submerging (dipping) the needle of the tattoo machine can lead to cross-contamination concerns. Additionally, the process of repeatedly submerging (dipping) the needle of the tattoo machine amounts to a significant amount of time during the course of a given tattoo procedure.

Various other solutions exist in the prior art that attempt to solve the aforementioned problems. For instance, U.S. Pat. No. 6,505,530B2 discloses an ink application device for tattooing or for making permanent makeup, which includes a handle, a needle drive, a needle, a needle nozzle, and a protective cap. The device further includes at least two modules releasably connected to one another. One of the modules is formed as a reusable base module with an integrated needle drive, and the other module is a sterilized module in which all components of the manual device capable of being infected by the bodily fluids of a customer are integrated. The device can be constructively divided into two or three separate modules: a basic module, a hygienic module, and an optional ink module. The ink quantity of approximately 0.5 to 2.0 ml provided is usually sufficient for one tattoo. If further ink is required for larger tattoo applications, the empty ink cartridge can easily be replaced by a new, sterile cartridge without the risk of contamination or infection.

The existing solutions related to ink refilling in tattoo machines are ineffective, complex in use, consume a lot of time, are costly, could lead to contamination, do not provide accurate and metered quantities of ink, and are inefficient in use. There is a need for an effective and efficient solution that solves the aforementioned problem by providing a tattoo machine that includes a removable ink bottle that stores a predetermined quantity of ink and an ink dispensing mechanism to dispense metered (accurate) quantity of ink from the removable ink bottle to the tattoo needle in a controlled and contact-less manner.

### SUMMARY

An object of the present invention is to provide a tattoo machine that can increase both the speed and accuracy of the tattoo-making process.

An object of the present invention is to provide a tattoo machine that eliminates the problem of cross-contamination of fluid.

An object of the present invention is to provide a tattoo machine that can be used several times during the lifetime of the tattoo machine.

An object of the present invention is to reduce the amount of time it takes to perform a tattoo procedure.

An object of the present invention is to provide a tattoo machine that can dispense a metered quantity of ink from a removable ink bottle to a tattoo needle in a controlled and contactless manner.

An object of the present invention is to provide a wireless handheld tattoo machine that allows the user to continuously apply a tattoo with a high degree of control, to function in a similar manner as an ideal tattoo gun.

An object of the present invention is to provide a tattoo machine that is configured to improve the efficiency of the standard tattoo-making process by eliminating the dipping of the tattoo needle in an ink reservoir.

An object of the present invention is to provide a peristaltic pump for the tattoo machine that can accurately deliver the small metered quantity of fluid to the desired surface in real-time, rendering them highly effective for microfluidics and contamination-free operations such as fluid supply in the tattoo machine.

While the way that the present disclosure addresses the disadvantages of the prior art will be discussed in greater detail below, in general, the present disclosure provides a tattoo machine that comprises an external casing that includes a top cover and a bottom cover. A sleeve partially encircles the bottom cover, wherein the sleeve is connected with the bottom cover. A removable fluid bottle is removably positioned on the top of the sleeve. A fluid dispensing mechanism comprises a tubing and a peristaltic pump. A tattoo needle is slidably disposed of in a tattoo nozzle. The fluid dispensing mechanism is configured to dispense a metered quantity of fluid from the removable fluid bottle to the tattoo needle in a controlled and contactless manner. A driving mechanism is configured to selectively operate the pump and enable longitudinal stroke motion for the tattoo needle.

Embodiments of the present invention disclose a tattoo machine comprising: an external casing that includes a top cover, and a bottom cover; a sleeve partially encircling the bottom cover, wherein the sleeve is connected with the bottom cover; a removable fluid bottle removably positioned on the top of the sleeve; a fluid dispensing mechanism connected to the bottom cover, wherein the fluid dispensing mechanism comprises a tubing, and a pump; a tattoo nozzle extending from the bottom of the bottom cover and fluidly connected to the fluid dispensing mechanism; a tattoo needle slidably disposed of in the tattoo nozzle; and a driving mechanism configured to selectively operate the pump and further enable longitudinal stroke motion for the tattoo needle.

In another embodiment, a visual charging indicator to indicate available battery power for usage and an on/off switch to activate/deactivate the tattoo machine.

In another embodiment, the sleeve includes a fluid passage and a fluid outlet hole that are collectively configured to provide passage of fluid for the tubing from the fluid outlet of the removable fluid bottle to a fluid dispensing mechanism and vice versa.

In another embodiment, the tattoo nozzle comprises a fluid supply collar, a rinse hole, a nozzle outlet; and wherein the tattoo needle comprises a needle overhang that projects outside the nozzle outlet when the tattoo needle is not undergoing longitudinal stroke motion.

In another embodiment, the tattoo nozzle comprises a fluid supply collar, a rinse hole, a nozzle outlet; and wherein the tattoo needle lies completely inside the nozzle outlet when the tattoo needle is not undergoing longitudinal stroke motion.

In another embodiment, the tubing is essentially bent in the form of a circle-shaped portion and the pump controls the fluid flow flowing through the tubing by enabling compression of the circle-shaped portion using the pump.

In another embodiment, the tubing is made of a flexible material comprising at least one of: silicon, rubber such as natural rubber, nitrile butadiene rubber (NBR), chlorosulphonated polyethylene rubber (CSM), ethylene-propylene-diene monomer (EPDM) and/or combinations thereof.

In another embodiment, the pump is a peristaltic pump.

In another embodiment, the pump is selected from a group comprising: a piezoelectric pump, a syringe pump, a diaphragm pump, an electrostatic pump, an electromagnetic pump, an electroosmotic pump, and a capillary pump.

In another embodiment, a battery ejector cap screwably coupled to the top end of the top cover, wherein the battery ejector cap is configured to enable the removal and replacement of a power source of the driving mechanism.

In another embodiment, a rotary dial is mechanically connected to the driving mechanism to enable longitudinal displacement of the driving mechanism in a predefined range within the external casing to further enable adjustment of a needle overhang (A) of the needle.

In another embodiment, the driving mechanism includes a first drive motor, a power source, a second drive motor for selectively operating the pump, and a transmission unit; wherein the transmission unit is configured to convert the rotational motion of the first drive motor into the translational motion of a tattoo needle; wherein the transmission unit includes an eccentric drive that includes an eccentric rotary cam, a stroke adjuster, a crank, and a sliding bar.

In another embodiment, the driving mechanism includes a controller to control the operation of the pump, and the driving mechanism wherein the controller includes an accelerometer for drop sensing to stop the functioning of the tattoo machine if low G-force is detected due to accidental falling of the tattoo machine.

In another embodiment, the tattoo machine is configured to be selectively deployed in either: a manual mode, a semi-automatic mode, or a fully automatic mode by controlling the operation of the pump and controlling the stroke length and stroke speed of the tattoo needle.

In another embodiment, the driving mechanism includes a first drive motor, a linkage mechanism, a power source, and a transmission unit; and the driving mechanism is configured for operating the pump and enabling longitudinal stroke motion for the tattoo needle.

In another embodiment, the tattoo machine is further configured to flush the fluid from the removable fluid bottle.

These and other features and advantages of the present invention will become apparent from the detailed description below, in light of the accompanying drawings.

### BRIEF DESCRIPTION OF DRAWINGS

A more complete understanding of the present disclosure may be derived by referring to the detailed description and claims when considered in connection with the figures, wherein like reference numerals refer to similar elements throughout the figures, and

FIG. 1 illustrates a first front perspective view of a tattoo machine, according to an embodiment of the present invention.

FIG. 2 illustrates a second front perspective view of the tattoo machine of FIG. 1.

FIG. 3 illustrates a front view of the tattoo machine of FIG. 1.

FIG. 4 illustrates a front sectional view of the tattoo machine of FIG. 1.

FIG. 5 illustrates a bottom view of the tattoo machine of FIG. 1.

FIG. 6 illustrates a front perspective view of a tattoo machine of FIG. 1, wherein the bottom cover, tubing, and the sleeve are not shown for the sake of simplicity and ease of understanding.

FIG. 7 illustrates a first-front perspective view of a fluid dispensing mechanism of the tattoo machine of FIG. 1.

FIG. 8 illustrates a second front perspective view of a fluid dispensing mechanism of the tattoo machine of FIG. 1.

FIG. 9 illustrates a sectional view of a tattoo machine of FIG. 1, wherein the bottom cover and the tattoo needle are not shown for the sake of simplicity and ease of understanding.

FIG. 10 illustrates a perspective view of the driving mechanism of the tattoo machine of FIG. 1.

### DETAILED DESCRIPTION

The following description is of exemplary embodiments of the invention only and is not intended to limit the scope, applicability, or configuration of the invention. Rather, the following description is intended to provide a convenient illustration for implementing various embodiments of the invention. As will become apparent, various changes may be made in the function and arrangement of the elements described in these embodiments without departing from the scope of the invention as set forth herein. It should be appreciated that the description herein may be adapted to be employed with alternatively configured devices having different shapes, components, attachment mechanisms, and the like and still fall within the scope of the present invention. Thus, the detailed description herein is presented for purposes of illustration only and not for limitation.

Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

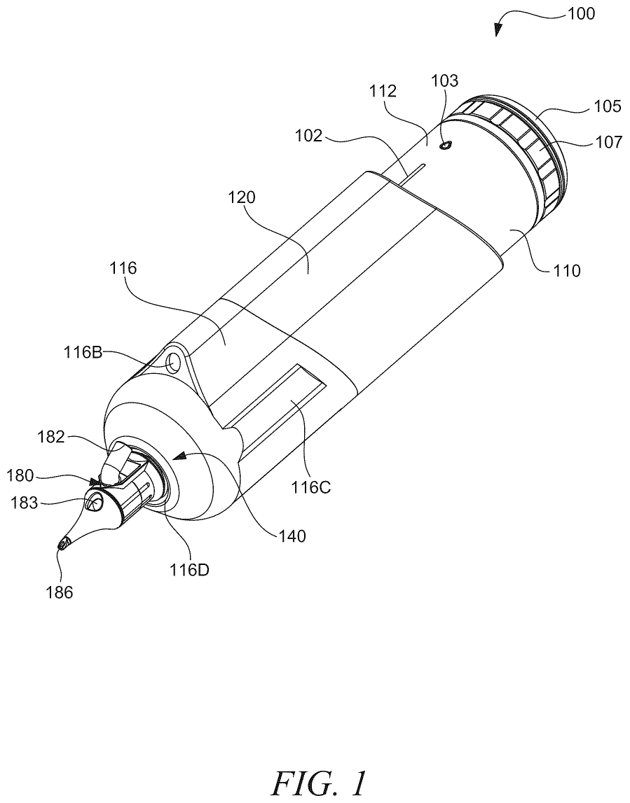

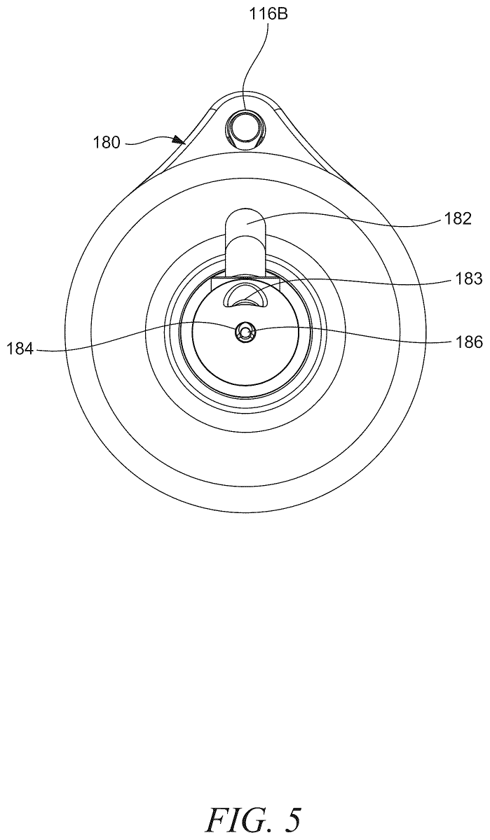

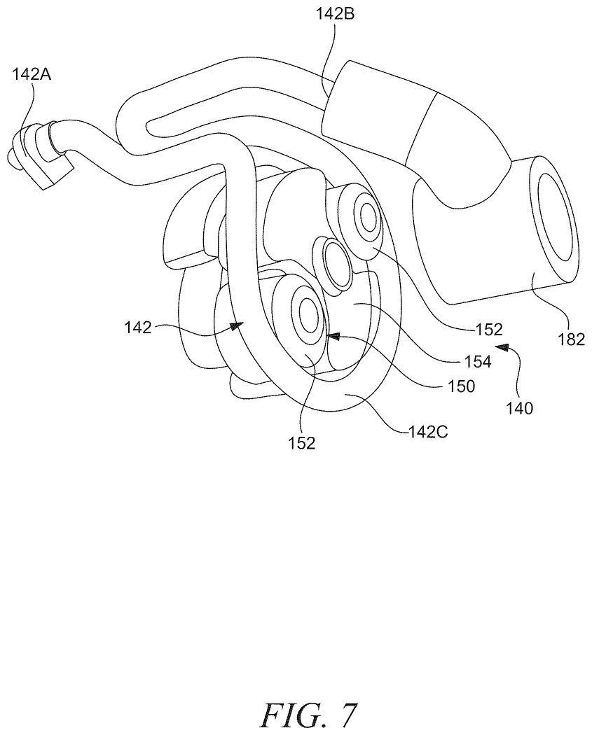

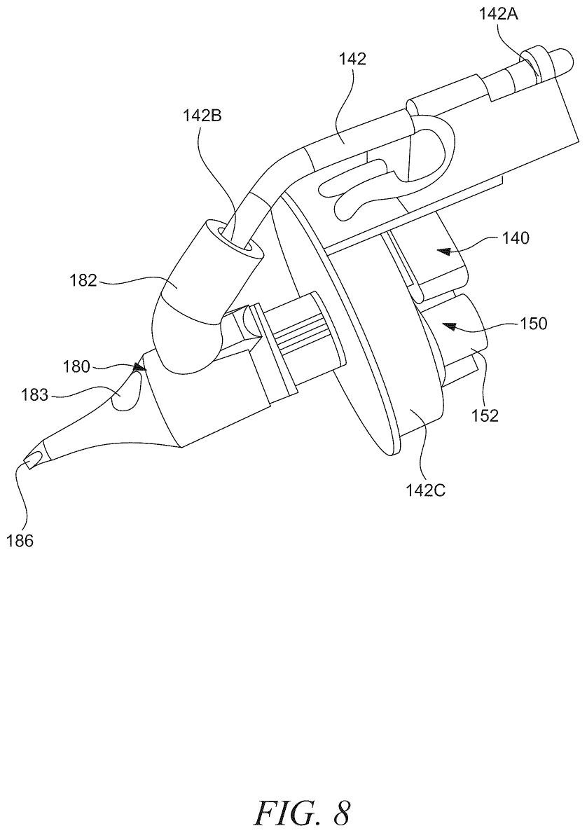

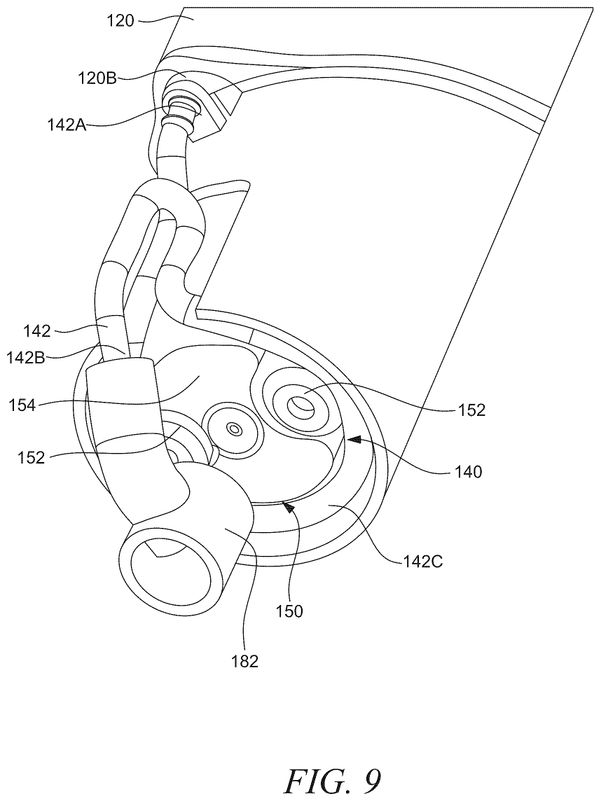

The tattoo machine will now be described with reference to the accompanying drawings, particularly FIGS. 1-10.

Reference is initially made to FIGS. 1-5 that illustrates a first front perspective view, a second front perspective view, a front view, a front sectional view, and a bottom view of a tattoo machine 100 respectively, according to an embodiment of the present invention. The tattoo machine 100 comprises an external casing 110 that is substantially cylindrical in shape and encloses (houses) various internal components associated with the tattoo machine 100 that will be described in greater detail in below description. As seen in FIGS. 1-5, a tubing 142 is not shown for the sake of simplicity and ease of understanding, wherein the tubing 142 will be described in greater detail in the below description.

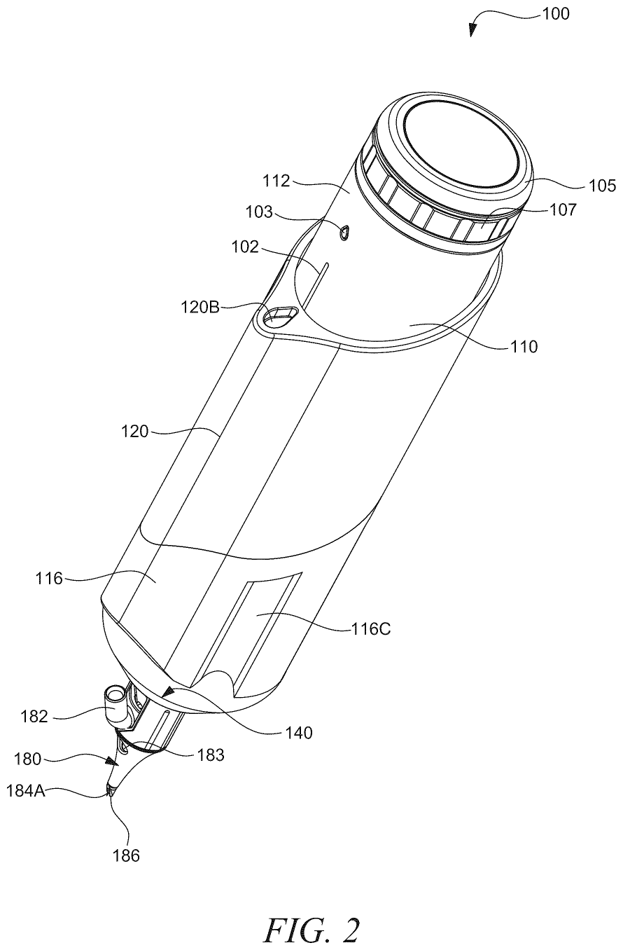

The external casing 110 includes a top cover 112, and a bottom cover 114. A sleeve 116 partially encircles the bottom cover 114 and the sleeve 116 is connected with the bottom cover 114. In an embodiment as seen in FIGS. 1-5, the sleeve 116 is substantially cylindrical in shape. However, in various other embodiments, the sleeve 116 may have other shapes, such as but not limited to: cuboidal, cubical, hexagonal and so on. The top cover 112 and the bottom cover 114 are removably connected by a connection mechanism such as but not limited to: screw arrangement, snap-fit arrangement and so on to form (define) a cavity that encloses various internal components/mechanisms associated with the tattoo machine 100, wherein the various internal components/mechanisms can include a fluid dispensing mechanism 140 and a driving mechanism 160, the entirety of which will be described in greater detail in below description. In an embodiment, as seen in FIG. 1 and FIG. 2, the tattoo machine 100 includes a visual charging indicator 102 to indicate available battery power for usage and an on/off switch 103 to activate/deactivate the tattoo machine 100.

A removable fluid bottle 120 is removably positioned on the top of the sleeve 116 and the removable fluid bottle 120 partially encircles the top cover 112. The removable fluid bottle 120 is essentially a double-walled cylindrical container that stores a predetermined quantity of fluid of known color such as but not limited to ink of various color, pigment, water, air, and so on. The removable fluid bottle 120 includes a fluid outlet 120A and a fluid inlet 120B. The sleeve 116 acts as a shoulder stop and keeps the removable fluid bottle 120 in place and further prevents the removable fluid bottle 120 from sliding down further relative to the bottom cover 114. Further, the sleeve 116 includes a fluid passage 116A and a fluid outlet hole 116B that are collectively configured to provide passage of fluid for the tubing 142 from the fluid outlet 120A of the removable fluid bottle 120 to a fluid dispensing mechanism 140 and vice versa that will be described in greater detail in below description.

In an exemplary embodiment, the tattoo machine 100 is configured to flush (remove) the fluid (contents) of the removable fluid bottle 120. The flushing of the fluid from the removable fluid bottle 120 is important for maintaining color accuracy and preventing cross-contamination. The flushing of the fluid from the removable fluid bottle 120 enables the tattoo machine 100 to remove substantially all of the undesired old fluid (pigment) from the tattoo machine 100 and prepare the removable fluid bottle 120 to receive new desired fluid (pigment).

The sleeve 116 further includes a grip portion 116C that is configured to be gripped by the hands of the user. The sleeve 116 further includes a bottom hole 116D that is dimensioned to allow a tattoo nozzle 180 to pass therethrough, wherein the tattoo nozzle 180 will be described in greater detail in the below description. The tattoo nozzle 180 extends from the bottom of the bottom cover 114. A tattoo needle 184 is slidably disposed of in the tattoo nozzle 180.

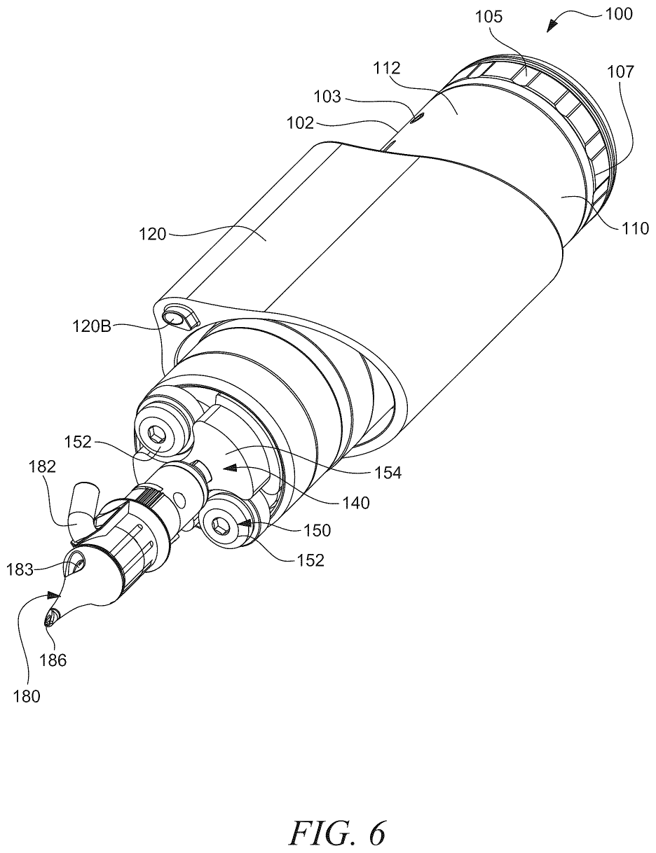

Referring to FIGS. 6-9, the fluid dispensing mechanism 140 comprises a tubing 142, and a pump 150. A first end 142A of the tubing 142 fluidly connects to a fluid outlet 120A of the removable fluid bottle 120. Further, an opposite second end 142B of the tubing 142 fluidly connects to a fluid supply collar 182 of a tattoo nozzle 180. Further, the tubing 142 is essentially bent in the form of a circle, herein designated as the circle-shaped portion 142C. The pump 150 controls the fluid flow flowing through the tubing 142 by enabling compression of the circle-shaped portion 142C using the pump 150, wherein the tubing 142 is made of a flexible material such as but not limited to: silicon, rubber such as natural rubber, nitrile butadiene rubber (NBR), chlorosulphonated polyethylene rubber (CSM), ethylene-propylene-diene monomer (EPDM) and so on. According to an embodiment of the present invention, the tubing 142 can be permanently fixed in the external casing 110. According to another embodiment of the present invention, the tubing 142 may be removable from the external casing 110. The pump 150 enables the fluid to move from the fluid outlet 120A of the removable fluid bottle 120 to the fluid supply collar 182 of a tattoo nozzle 180 through the tubing 142. Further, the pump 150 can be selectively enabled to move the fluid to flow in reverse direction from the tubing 142 into the fluid outlet 120A of the removable fluid bottle 120 to assist in flushing of the fluid (contents) of the removable fluid bottle 120.

In an embodiment as seen in FIGS. 6-9, the pump 150 is a peristaltic pump 150 that includes a plurality of rotation rollers 152, and a central lobe 154. The central lobe 154 is configured to rotate on its fixed longitudinal axis which will be described later in greater detail in the below description. The plurality of rotation rollers 152 follows a circular orbital motion that corresponds to the circle-shaped portion 142C of the tubing 142. The circular orbital motion of the plurality of rotation rollers 152 compresses the circle-shaped portion 142C of the tubing 142 to thereby provide a pumping effect for the fluid disposed of in the compressed tubing 142. The central lobe 154 is rotationally driven by a driving mechanism 160 that will be described in greater detail in the below description. The peristaltic pump 150 dispenses a continuous metered (accurate) quantity of fluid to the fluid supply collar 182 of the tattoo nozzle 180 in a contactless manner and provides the advantage of no cross-contamination of fluid. Further, the peristaltic pump 150 can accurately deliver the small metered quantity of fluid to the desired surface in real-time, rendering them highly effective for microfluidics and contamination-free operations such as fluid supply in tattoo machine 100.

In an exemplary embodiment, an old removable fluid bottle 120, previously used by a first user is removed from the tattoo machine 100 and the first new removable fluid bottle 120 is installed on the tattoo machine 100, wherein the first new removable fluid bottle 120 comprises cleaning agent such as but not limited to: water, soap solution, detergent solution and so on. Afterward, the tattoo machine 100 is switched on, and the cleaning agent of the first new removable fluid bottle 120 cleans the tubing 142 by rinsing effect. Now, the tattoo machine 100 is ready for use for making tattoos on the desired surface such as the body of the second user. Afterward, a second new removable fluid bottle 120 is installed on the tattoo machine 100 for use on the body of the second user, and this similar process is repeated for the next user and so on.

In another embodiment (not shown in figures), the pump 150 is any device capable of accurately pumping fluid in small quantities, such as but not limited to: piezoelectric pump, syringe pump, diaphragm pump, electrostatic pump, electromagnetic pump, electro-osmotic pump, capillary pump, and so on.

Further, the tattoo machine 100 includes a battery ejector cap 105 screwably coupled to the top end of the top cover 112, wherein the battery ejector cap 105 is configured to enable removal and replacement of the power source 168 (cell/battery) in a manner similar to the cell replacement of a flashlight. The battery ejector cap 105 can include any conventional components, such as but not limited to: wave spring, torsion spring, coil spring, twist lock, corkscrew, and so on. Further, the tattoo machine 100 may include a rotary dial 107. The rotary dial 107 may be mechanically connected to the driving mechanism 160 by any connection means such as but not limited to: threaded connection, sliding arm, gear linkage, chain linkage, and so on such that the rotary dial 107 may be rotated to enable longitudinal displacement (up-down motion) of the driving mechanism 160 in a predefined range (typically 0-10 millimeter) within the external casing 110 to enable adjustment of the needle overhang 184A, that will be described in greater detail in below description.

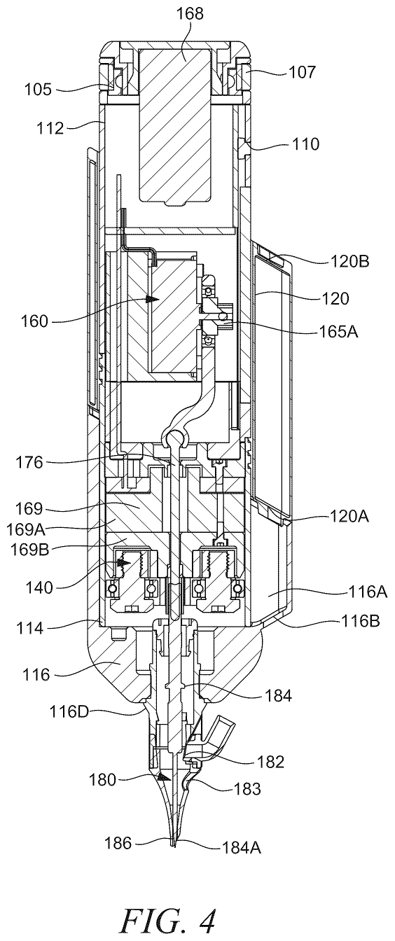

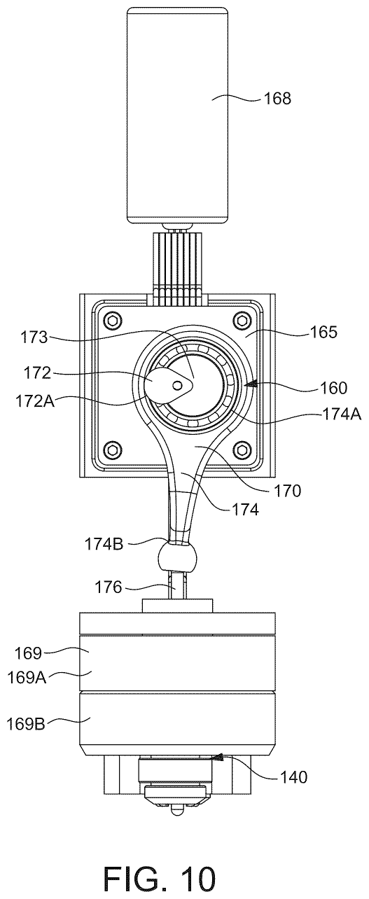

FIG. 10 illustrates a perspective view of the driving mechanism 160 of the tattoo machine 100. Referring to FIG. 4 and FIG. 10, the driving mechanism 160 serves dual purposes that include operating the pump 150 and enabling longitudinal stroke motion for the tattoo needle 184. The driving mechanism 160 includes a first drive motor 165, a power source 168, a second drive motor 169, and a transmission unit 170. The power source 168 is configured to provide electric power to the first drive motor 165 and the second drive motor 169. The power source 168 can include but is not limited to: cell/battery and so on. The first drive motor 165 includes a motor output shaft 165A and the first drive motor 165 can be any electric motor capable of converting electric energy of power source 168 into rotational (mechanical) energy and the first drive motor 165 may include but not limited to: direct current (DC) motor, alternating current (AC) motor, servomotor, stepper motor, universal motor and so on. In an embodiment as seen in FIG. 10, the first drive motor 165 is a direct current (DC) brushless motor with an encoder for speed control feedback. In an embodiment (not shown in figures), the power source 168 is configured to be electrically charged by a charging station (device) (not shown in figures). In another embodiment (not shown in figures), the power source 168 is configured to be electrically charged by using a power socket (not shown in figures) that is connected to a conventional electricity (utility) supply line.

Referring to FIG. 4 and FIG. 10, the second drive motor 169 is configured to selectively operate pump 150 by enabling the rotation of the plurality of rotation rollers 152. The second drive motor 169 includes a stator 169A that is connected to the bottom cover 114, and a rotor 169B rotates to further rotate the central lobe 154 and/or the plurality of rotation rollers 152. The second drive motor 169 can be any electric motor capable of converting the electric energy of power source 168 into rotational (mechanical) energy of the rotor 169B and the second drive motor 169 can include but not be limited to gimbal motor, direct current (DC) motor, alternating current (AC) motor, servomotor, stepper motor, universal motor and so on. In an embodiment as seen in FIG. 10, the second drive motor 169 is a gimbal motor.

The transmission unit 170 is configured to convert the rotational motion of the motor output shaft 165A into the translational motion of the tattoo needle 184. The transmission unit 170 includes an eccentric drive 170 that includes an eccentric rotary cam 172, a stroke adjuster 173, a crank 174, and a sliding bar 176. The eccentric rotary cam 172 is rotationally connected to the motor output shaft 165A. The eccentric rotary cam 172 includes a cam surface 172A that is configured to contact an interior circular periphery 174A of the crank 174. The stroke adjuster 173 is essentially a circular-shaped disk of a certain diameter. The stroke adjuster 173 and/or the eccentric rotary cam 172 can be replaced by another stroke adjuster 173 and/or the eccentric rotary cam 172 having a different diameter (dimension), thereby enabling the stroke length adjustment of the sliding bar 176 and hence, the tattoo needle 184. The crank 174 comprises an elongated end portion 174B that is connected to the sliding bar 176. The sliding bar 176 is hollow-shaped in nature and is configured to enable stroke (longitudinal) motion of the tattoo needle 184. The sliding bar 176 is made of any material having sufficient strength such as but not limited to: polyamide, nylon, aluminum, steel, glass-reinforced nylon, and so on.

The driving mechanism 160 includes a controller (not shown in the figures) that selectively controls the operation of the pump 150, the first drive motor 165, and the second drive motor 169. Further, the controller (not shown in the figures) may be wirelessly connected to a remote device such as but not limited to: a smartphone, tablet, desktop computer, laptop, pager, and so on by using an application software. The user can open the application software (app) to control the operation of the tattoo machine 100 by using the controller (not shown in the figures). In another embodiment (not shown in the figures), the controller (not shown in the figures) is connected to a foot pedal (foot switch) (not shown in the figures) by using a primary wireless communication protocol already known in the art wherein the wireless communication protocol may include but not limited to: IEEE 802.15.1 (Bluetooth), wireless fidelity (WIFI), radio communication, infrared communication, ZigBee, wireless local area network (WLAN), cellular network and so on. In another embodiment (not shown in the figures), a secondary infrared (IR) control channel is formed between the foot pedal (foot switch) (not shown in the figures) and the controller (not shown in the figures). Further, the foot pedal (foot switch) (not shown in the figures) may include a user interface that electronically connects to the user interface of the application software. The user interface of the application software (app) and user interface of the foot pedal is synchronized such that the user interface of the application software (app) and user interface of the foot pedal mirror one another. The user interface may allow users to interact with electronic devices and the user interface may include but not limited to: a graphical user interface (GUI), multimedia user interface (MUI), and so on.

Further, the controller (not shown in the figures) includes an accelerometer for drop sensing to stop the functioning of the tattoo machine 100 if low G-force is detected due to accidental falling of the tattoo machine 100.

Further, the driving mechanism 160 includes necessary electronic components necessary for the operation of the tattoo machine 100 such as but not limited to: printed circuit board, wiring, cables, switches, resistors, sensors, Bluetooth circuitry, transformer, rectifier, filters and regulators, universal serial bus (USB) interface (port) and so on.

In various embodiments, the tattoo machine 100 is configured to be selectively deployed in either one of the multiple modes: that is, a manual mode, a semi-automatic mode, and a fully automatic mode depending on the requirements of the user. Depending on the mode selected from the manual mode, the semi-automatic mode, and the fully-automatic mode, the user and/or controller (not shown in figures) of the driving mechanism 160 can either control (select) the operational parameters of the tattoo machine 100 by controlling the speed and revolutions of the pump 150 and further, control stroke length and stroke speed of the tattoo needle 184.

In another embodiment (not shown in figures), the transmission unit 170 may include any mechanism/components capable of converting the rotational motion of the motor output shaft 165A into the translational motion of the tattoo needle 184. The mechanism/components can include but are not limited to the scotch yoke mechanism, slider crank mechanism, cam follower mechanism, crankshaft, gear drive, chain drive, and so on.

In another embodiment (not shown in figures), the driving mechanism 160 includes only one motor that is a first drive motor 165, a linkage mechanism (not shown in figures), a power source 168, and a transmission unit 170. The first drive motor 165 in conjugation with the linkage mechanism (not shown in figures) serves dual purposes that include operating the pump 150 and enabling longitudinal stroke motion for the tattoo needle 184. The transmission unit 170 is configured to convert the rotational motion of the motor output shaft 165A into the translational motion of the tattoo needle 184. The linkage mechanism (not shown in figures) can include any mechanism capable of transmitting the rotational motion of the motor output shaft 165A of the first drive motor 165 into the rotation of the plurality of rotation rollers 152.

Referring to FIG. 4, the tattoo nozzle 180 comprises a tattoo needle 184 that is connected to the sliding bar 176 by a suitable connection means. The tattoo needle 184 is slidably arranged in a tattoo nozzle 180 and the tattoo needle 184 is capable of piercing the desired surface such as the skin of the user. The tattoo nozzle 180 is configured to dispense a metered (accurate) quantity of fluid to the desired surface such as the skin of the user. The tattoo nozzle 180 comprises a fluid supply collar 182, a rinse hole 183, and a nozzle outlet 186. The fluid supply collar 182 is fluidly connected to a tubing 142 and the fluid supply collar 182 is substantially cylindrically shaped to allow fluid coming from the tubing 142 to distribute in the fluid supply collar 182 as well as to allow longitudinal stroke movement of the tattoo needle 184. The fluid passing through the fluid supply collar 182 is dispensed (deposited) on the tattoo needle 184 and then is delivered to the desired surface such as the body of a user. The rinse hole 183 enables cleaning of the tattoo nozzle 180 with a suitable cleaning agent (fluids). The nozzle outlet 186 dispenses accurate quantities of the fluid to the surface.

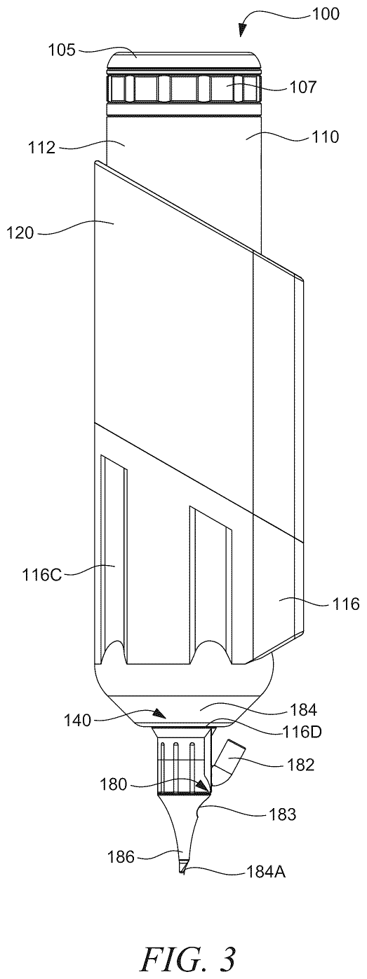

In an embodiment as seen in FIG. 2 and FIG. 3, a small portion of tattoo needle 184 projects outside the nozzle outlet 186 when the tattoo needle 184 is not undergoing longitudinal stroke motion (herein referred to as the retracted position of the tattoo needle 184). The projecting portion of the tattoo needle 184 is designated as needle overhang 184A. In another embodiment (not shown in figures), the tattoo needle 184 lies completely inside the nozzle outlet 186 when the tattoo needle 184 is not undergoing longitudinal stroke motion (herein referred to as retracted position). Further, the tattoo needle 100 provides a customization option to adjust the length of the needle overhang 184a in the retracted position. Further, the tattoo machine 100 can be customized to enable tattoo needle 184 to completely lie (otherwise referred to as negative needle overhang 184A) inside the nozzle outlet 186 when the tattoo needle 184 is in the retracted position.

The various components and parts of the various embodiments of the tattoo machine 100 of the present invention are similar and interchangeable. Further, it should be understood that the components of the tattoo machine 100 can be made of any material and any size depending on the type of tattoo machine 100. Further, the shape of the tattoo machine 100 can be modified depending on the requirements of the tattoo machine 100. Further, it should be obvious that the tattoo machine 100, in particular, moving parts of the tattoo machine 100 includes various components essential for the construction and/or operation of the tattoo machine 100 wherein the components can include but are not limited to bearings, screws, bushings, pins, rivets, threaded couplings and so on.

Finally, while the present invention has been described above with reference to various exemplary embodiments, many changes, combinations, and modifications may be made to the exemplary embodiments without departing from the scope of the present invention. For example, the various components may be implemented in alternative ways. These alternatives can be suitably selected depending upon the particular application or in consideration of any number of factors associated with the operation of the device. In addition, the techniques described herein may be extended or modified for use with other types of devices. These and other changes or modifications are intended to be included within the scope of the present invention.

What is claimed is:

1. A tattoo machine (100) comprising:
an external casing (110);
a sleeve (116) partially encircling the external casing (110), wherein the sleeve (116) is connected with the external casing (110);
a removable fluid bottle (120) removably positioned on the top of the sleeve (116);
a fluid dispensing mechanism (140) connected to the external casing (110), wherein the fluid dispensing mechanism (140) comprises a tubing (142), and a pump (150);
a tattoo nozzle (180) extending from the bottom of the external casing (110) and fluidly connected to the fluid dispensing mechanism (140);
a tattoo needle (184) slidably disposed of in the tattoo nozzle (180); and
a driving mechanism (160) configured to operate the pump (150) and further enable longitudinal stroke motion for the tattoo needle (184).

2. The tattoo machine (100) according to claim 1, wherein the external casing (110) includes a top cover (112) and a bottom cover (114); and the sleeve (116) partially encircles the bottom cover (114); wherein the sleeve (116) is further connected with the bottom cover (114); and the fluid dispensing mechanism (140) is connected to the bottom cover (114).

3. The tattoo machine (100) according to claim 1, further comprising a visual charging indicator (102) to indicate available battery power for usage and an on/off switch (103) to activate/deactivate the tattoo machine (100).

4. The tattoo machine (100) according to claim 1, wherein the sleeve (116) includes a fluid passage (116A) and a fluid outlet hole (116B) that are collectively configured to provide passage of fluid for the tubing (142) from the fluid outlet (120A) of the removable fluid bottle (120) to a fluid dispensing mechanism (140) and vice versa.

5. The tattoo machine (100) according to claim 1, wherein the tattoo nozzle (180) comprises a fluid supply collar (182), a rinse hole (183), a nozzle outlet (186); and wherein tattoo needle (184) comprises a needle overhang (184A) that projects outside the nozzle outlet (186) when the tattoo needle (184) is not undergoing longitudinal stroke motion.

6. The tattoo machine (100) according to claim 1, wherein the tattoo nozzle (180) comprises a fluid supply collar (182), a rinse hole (183), a nozzle outlet (186); and wherein the tattoo needle (184) lies completely inside the nozzle outlet (186) when the tattoo needle (184) is not undergoing longitudinal stroke motion.

7. The tattoo machine (100) according to claim 1, wherein the tubing (142) is substantially bent in the form of a circle-shaped portion (142C) and the pump (150) controls the fluid flow flowing through the tubing (142) by enabling compression of the circle-shaped portion (142C) using the pump (150).

8. The tattoo machine (100) according to claim 1, wherein the tubing (142) is made of a flexible material comprising at least one of: silicon, rubber such as natural rubber, nitrile butadiene rubber (NBR), chlorosulphonated polyethylene rubber (CSM), ethylene-propylene-diene monomer (EPDM) and/or combinations thereof.

9. The tattoo machine (100) according to claim 1, wherein the pump (150) is a peristaltic pump (150).

10. The tattoo machine (100) according to claim 1, wherein the pump (150) is selected from a group comprising of: piezoelectric pump, syringe pump, diaphragm pump, electrostatic pump, electromagnetic pump, electro osmotic pump, and capillary pump.

11. The tattoo machine (100) according to claim 1, further comprising a battery ejector cap (105) screwably coupled to the top end of the external casing (110), wherein the battery ejector cap (105) is configured to enable removal and replacement of a power source (168) of the driving mechanism (160).

12. The tattoo machine (100) according to claim 1, further comprising a rotary dial (107) that is mechanically connected to the driving mechanism (160) to enable longitudinal displacement of the driving mechanism (160) in a predefined range within the external casing (110) to further enable adjustment of a needle overhang (184A) of the needle (184).

13. The tattoo machine (100) according to claim 1, wherein the driving mechanism (160) includes a first drive motor (165), a power source (168), a second drive motor (169) for selectively operating the pump (150), and a transmission unit (170); wherein the transmission unit (170) is configured to convert the rotational motion of the first drive motor (165) into the translational motion of a tattoo needle (184); wherein the transmission unit (170) includes an eccentric drive (170) that includes an eccentric rotary cam (172), a stroke adjuster (173), a crank (174), and a sliding bar (176).

14. The tattoo machine (100) according to claim 1, wherein the driving mechanism (160) includes a controller to control the operation of the pump (150) and the driving mechanism (160) wherein the controller includes an accelerometer for drop sensing to stop the functioning of the tattoo machine (100) if low G-force is detected due to accidental falling of the tattoo machine (100).

15. The tattoo machine (100) according to claim 1, wherein the tattoo machine (100) is configured to be selectively deployed in either one of: a manual mode, a semi-automatic mode, and a fully automatic mode by controlling the operation of the pump (150) and controlling stroke length and stroke speed of the tattoo needle (184).

16. The tattoo machine (100) according to claim 1, wherein the driving mechanism (160) includes a first drive motor (165), a linkage mechanism, a power source (168), and a transmission unit (170); and the driving mechanism (160) is configured for operating the pump (150) and enabling longitudinal stroke motion for the tattoo needle (184).

17. The tattoo machine (100) according to claim 1, wherein the tattoo machine (100) is further configured to flush the fluid from the removable fluid bottle (120).
